(19) United States
(12) Patent Application Publication
(10) Pub. No.: US 2025/0033055 A1
(43) Pub. Date: Jan. 30, 2025

(54) SEQUENCING SYSTEMS AND METHODS UTILIZING THREE- DIMENSIONAL SUBSTRATES

(71) Applicant: MGI Tech Co., Ltd., Shenzhen (CN)

(72) Inventors: Kee Tsz Woo, Sunnyvale, CA (US); Michelle Jarrell, San Jose, CA (US); Paul Lundquist, Oakland, CA (US); Jay Shafto, Mountain View, CA (US)

(21) Appl. No.: 18/294,719

(22) PCT Filed: Aug. 2, 2022
(86) PCT No.: PCT/CN2022/109588

§ 371 Date: Feb. 2, 2024

Related U.S. Application Data
(60) Provisional application No. 63/229,268, filed on Aug. 4, 2021.

(51) Int. Cl. B01L 3/00; C12Q 1/6874

(52) U.S. Cl. CPC B01L 3/5085; C12Q 1/6874; B01L 2200/12; B01L 2300/0893; B01L 2300/0896; B01L 2300/168

(57) ABSTRACT

A nucleic acid sequencing system may include a substrate including a three-dimensionally patterned surface. The three-dimensionally patterned surface may define nanowells each including a derivitized area for binding to nucleic acid template molecules. The nanowells may be 100 nm in diameter with 350 nm center-to-center spacing. The substrate may including reflective layers and plasmonically enhanced layers for increasing fluorescent signals during nucleic acid sequencing.

### CROSS-REFERENCE TO RELATED APPLICATIONS

This application claims the benefit of and priority to U.S. Provisional Application No. 63/229,268 filed on Aug. 4, 2021, which is hereby incorporated by reference in its entirety.

### RELATED FIELDS

This disclosure relates to systems for nucleic acid sequencing and other biochemical analyses.

### BACKGROUND

Nucleic acid sequencing includes numerous different costs, for example, costs related to the purchase and upkeep of the sequencing device, as well as the costs of reagents. Reducing the amount of time to produce the same amount of sequencing data and/or reducing the amount of reagents used compared to existing sequencing devices may reduce the overall costs of producing the sequencing data.

Some currently available sequencing systems detect sequencing events on an essentially rectangular 2-dimensional planar substrate of a flowcell. An objective of an optical detection system and the flowcell are moved relative to each other so that the field of view of the objective is passed over the substrate a plurality of times, wherein each pass images a portion of the substrate so that the entire substrate is imaged.

Due to the current imaging and binding technologies used with planar substrate, these systems have a limitation associated with the maximum density of individual nucleic acid sites possible on a planar substrate. Accordingly, there is a need to increase the density of individual nucleic acid sites compared to existing technologies used with planar substrates.

### BRIEF SUMMARY

This present technology relates to substrates used with systems for detecting sequencing events. The systems may be employed in, for example, sequencing nucleic acid molecules disposed on a substrate, wherein the substrate may include from millions to billions of individual recesses, each defining a nucleic acid site. The substrate may include a three-dimensional, i.e. non-planar surface, defining an array of the recesses, and may be referred to as a three-dimensionally patterned substrate. Each recess, also referred to as a nanowell may define an individual nucleic acid site for containing sequencing nucleic acid molecules during the sequencing events. The substrate may be moved relative to a field of view (FOV) of a detection system, for example an objective of an optical detection system, so that the FOV passes over the substrate in order to image the sequencing events in each recess. Advantages of the disclosed substrates used with systems for detecting sequencing events include allowing for closing spacing of nucleic acid sites compared to planar substrates due to the recesses preventing adjacent sequencing nucleic acid molecules from spreading toward each and cross-contaminating adjacent nucleic acid sites. Closer spacing of nucleic acid sites also allows for more nucleic acid sites in the FOV of the detection system and therefore may lead to improved throughput thereby creating significant cost savings as will be discussed herein.

### BRIEF DESCRIPTION OF THE DRAWINGS

FIGS. 1A-1C show an embodiment of a planar substrate.

FIGS. 2A-2C show an embodiment of substrate including nanowells.

FIGS. 3A-3C show an embodiment of substrate including photoresist defining nanowells.

FIG. 4 shows an embodiment of substrate including nanowells defined with curved bottom and sidewalls.

FIG. 5 shows an embodiment of substrate including nanowells defined with angled sidewalls.

FIGS. 6A-6C show a method of forming nanowells in substrate.

FIGS. 7A-7C show a method of forming nanowells in substrate.

FIGS. 8A and 8B show a method of forming nanowells in substrate.

FIGS. 9, and 10 show embodiments of multi-layer substrates including nanowells.

In accordance with common practice, the described features and elements are not drawn to scale but are drawn to emphasize features and elements relevant to the present disclosure.

### DETAILED DESCRIPTION

The present disclosure describes substrates used with sequencing detection systems that may be employed in detecting sequencing events. For example, a sequencing detection system may be an optical imaging system employed in sequencing for example, nucleic acids. In embodiments, the template nucleic acid molecules may be bound to, or otherwise disposed within the recesses, also referred to as nanowells, of a three-dimensionally patterned substrate and then imaged by a detection system, for example an optical imaging system.

There are many approaches to nucleic acid (e.g., DNA) sequencing. See, e.g., Kumar, K., 2019, “Next-Generation Sequencing and Emerging Technologies,” Semin Thromb Hemost 45 (07): 661-673. The most popular methods use arrays of discrete sites on a planar substrate. The arrays may include a large number of discrete sites (e.g., 100 million to 1 billion or more) on a single planar substrate. Typically the sites are small (e.g., characterized by a diameter or diagonal less than 1 micrometer, often less than 500 nanometers, and often in the range of 50 nanometers to 500 nanometers) and present at a density of more than ˜˜106 sites per cm². For example, the sites may have a diameter of 200 nm and a 700 nm center-to-center spacing. Nucleic acid templates are immobilized directly or indirectly at the individual sites on the planar substrate for sequencing. Generally each site contains a clonal population of template sequences, such as a DNA nanoball (Complete Genomics, Inc.) or PCR products or amplicons (Illumina, Inc.). For illustration and not limitation, in these approaches nucleic acid sequences are determined one base at a time over a series of sequencing “cycles.” Each cycle comprises (i) introducing reagents to each site on the array of immobilized template molecules; (ii) carrying out a series of biochemical or enzymatic reactions (“sequencing reactions”) simultaneously at the sites; (iii) detecting signals at each site (zero, one or more than one signal per site per cycle) which may be referred to as “image acquisition:”; and (iv) carrying out enzymatic, washing, or regeneration steps at each site on the array so that another sequencing cycle can be carried out. Without limitation the “signals” collected in (iii) may be optical signals, e.g., fluorescence or luminescence signals. The sequencing array of the planar substrate is usually contained in a “flow cell” through which primers, reagents, washes, etc. can be flowed. Typically a sequencing run consists of ˜400 cycles, which means that ˜400 or more imaging events, each involving acquiring signal individually from each of millions of sites is required. The speed and precision of image collection affects cost, efficiency, and sequencing data quality.

As used herein a “sequencing event” refers to emission of an optical signal (e.g., a fluorescence or luminescence signal) resulting from a sequencing process. An exemplary sequencing process is a cycle of a sequencing-by-synthesis process. In this approach, nucleotides are incorporated into a primer extension product (e.g. using reversible terminator nucleotides). In this approach, nucleotides can be labeled with, for example, a fluorescent dye or a source of a luminescence signal (e.g. luciferase or luciferase substrate). A luminescent signal includes chemiluminescence and bioluminescence. A nucleotide can be labeled directly with a fluorescent dye or a source of a luminescence signal or can be associated with an antibody, aptamer or other agent labeled with a signal generating moiety. In the process of sequencing a defined optical signal is produced at each site in an array by, for example, illumination of the fluorescent dye(s) with an excitation wavelength, and the signals and corresponding positions are recorded.

Although framed in the context of nucleic acid sequencing, it will be recognized that the devices and methods disclosed herein are not limited to nucleic acid sequencing uses. The devices and methods may be used, for example, for nucleic acid analysis other than sequencing (e.g., SNP analysis, real time PCR analysis) or for analysis of chemical or biochemical processes using substrates or analytes other than nucleic acids.

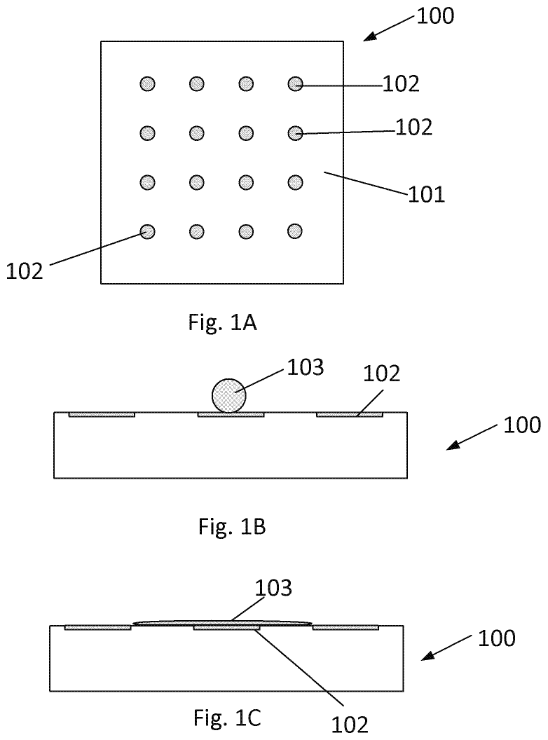

FIG. 1A shows a substrate 100, or a portion thereof. As shown, the substrate 100 includes a planar surface 101 including a plurality of derivitized areas 102 (“spots” or discrete spaced apart regions). The derivitized areas 102 may be positioned in a patterned array, for example in rows and columns as shown in FIG. 1A. The derivitized areas 102 may be adapted to contain nucleic acid template molecules. For example, the derivitized areas 102 may be defined by surface chemistry of the substrate 100 configured for binding to nucleic acid template molecules. Examples of surface chemistries for binding to nucleic acid template molecules include (3-Aminopropyl) trimethoxysilane (APTMS) CVD amination. The portions of the substrate around the derivitized areas 102 may include a surface chemistry configured to not bind to nucleic acid template molecules. The derivitized areas 102 may be substantially planar with a top surface of the substrate 100, including the non-binding surface around the derivitized areas, as shown in FIG. 1B. The processes of forming the derivitized area and non-binding surfaces, may include HMDS vapor deposition, photolithography, O2 plasma etching, aminosilanization (CVD), and resist striping. These processes may result in surface variations of the planar surface, for example a non-uniform and/or discontinuous portion of cross-linking of aminosilane and photoresist may form around the perimeter of the derivitized area, and may be referred to as “crust”. Portions of the non-uniform and/or discontinuous crust may have a height above a substantially planar surface of the derivitized area of about 5% of the diameter of the derivitized area, e.g. ˜10 nm height around a 200 nm diameter derivitized area. Due to the uniform and/or discontinuous characteristics of the crust, as well as the relative small height, e.g. <˜5%, an array of derivitized areas including a crust, as noted above, are considered to be a “planar substrate” as used herein. The example substrate 100, or portion therefore, is shown for illustrative purposes to include sixteen derivitized areas 102, in embodiments, a substrate may include more than 105, more than 106, more than 107 sites, more than 108 sites, more than 109 sites, or more than 1010 derivitized areas, such as from 105 to 1011 sites or 106 to 1010 derivitized areas. The derivitized areas may be regions of the substrate surface 101 derivitized to bind nucleic acid molecules (e.g., DNA nanoballs (DNBs), a template cluster produced by bridge amplification, or other templates).

FIG. 1B shows a side cross-sectional view of an example of nucleic acid molecules, specifically DNBs 103, binding to a derivitized area 102, between two other derivitized areas 102. The DNBs 103 may be introduced in a solution into a flowcell containing the substrate 100. Floating in the solution, the DNBs 103 may be substantially spherical, as shown in FIG. 1B. Once bound to the derivitized areas 102, the DNBs 103 tend to flatten out, as shown in FIG. 1C, and in the flattened configuration the DNBs may extend beyond derivitized areas 102 onto non-derivitized areas of the substrate 100. FIG. 1C shows an example amount of flattening and spreading of a DNBs for illustrative purposes, and in some embodiments degree of flattening and/or spreading may be more and/or less than shown. In some embodiments, the number of copies of the nucleic acid molecules of the DNBs 103 may be caused to increase after binding of the DNBs, and thus increase the volume of the already bound DNB. Increasing the volume of the bound DNBs may cause an outer perimeter of the flattened DNBs to spread even further away from the derivitized areas 102 onto non-derivitized areas of the substrate 100.

In embodiments, it is beneficial for the derivitized areas 102 to be spaced closely together which may result in a detection system being able to capture more sequencing events per FOV of the detection system and/or decrease the amount of reagents used during a sequencing process. However, due to the flattening of the DNBs on a planar substrate, if the spacing between adjacent derivitized areas 102 approaches the outer diameter of flattened DNBs, the DNBs may spread toward and over adjacent derivitized areas, as shown for example in FIG. 1C. The spreading to adjacent derivitized areas is referred to as non-specific binding. Non-specific binding can cause under loading and affect the signal intensity for sequencing, also referred to as cross-talk which can lead to corruption of sequencing data. For example, during imaging of sequencing events, a fluorescent signal from a first DNB bound to a first derivitized area may unintentionally be detected as being associated with an adjacent second derivitized area which the first DNB is spread toward due to the resolution of the detection system. The unintentional detection of a fluorescent signal from a DNB not bound to the derivitized area is referred to as cross-talk. Accordingly, embodiments of the present technology are directed toward substrates which reduce or eliminate cross-talk, while reducing the spacing between derivitized areas. The sizes of the DNBs when reducing the spacing between derivitized areas may remain the same or be smaller than DNBs used with planar substrates.

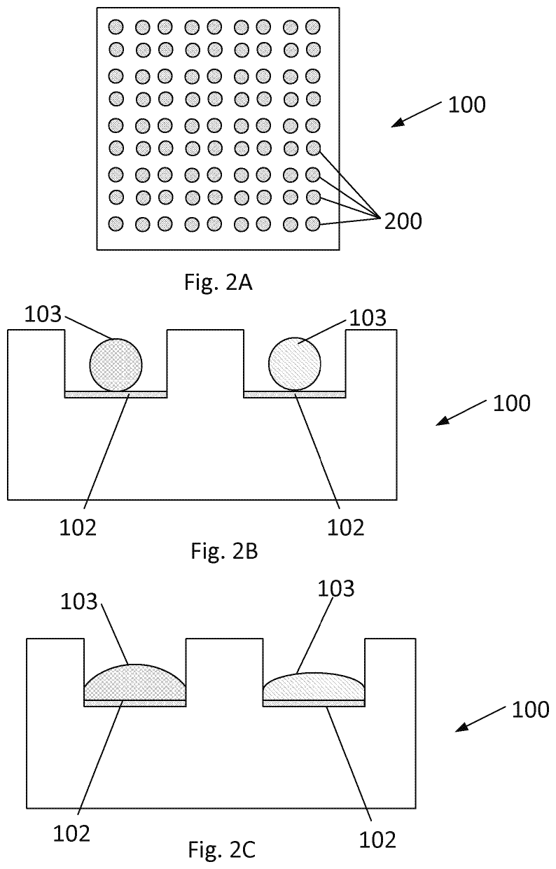

In some embodiments, the substrate 100 includes nanowells 200 for each derivitized area, for example as shown in FIGS. 2A-2C. FIG. 2A shows a top view of a substrate 100 including nanowells 200. The substrate 100 defines a top surface 201, and the nanowells 200 are recessed below the top surface 201 as shown in FIG. 2B. As used herein, “nanowell” refers to a well, i.e. a recess with all dimensions less than 1000 nanometers. As shown, the nanowells 200 may be positioned in an ordered array, similar to the ordered array of FIGS. 1A-1C. The nanowells may have diameter/width between 100 nm and 300 nm, for example 150 nm. The nanowells may be spaced center-to-center between 250 and 500 nm, for example 350 nm. The nanowells may have a depth of greater 25% of their diameter, for example a depth of 100-150 nm. The nanowells may have a volume of 5.0×10⁻¹³ to 8.5×10⁻¹³ mm³. A substrate may include an ordered array of nanowells of 150 nm diameters and 100 nm depths with 350 nm center-to-center spacing, resulting in edge-to-edge spacing of 200 nm. This arrangement of nanowells is beneficial compared to an ordered two-dimensional array of derivitized areas with 150 nm diameters with 350 nm center-to-center spacing since the two-dimensional array of derivitized areas would lead to cross-talk. The center-to-center spacing, and/or edge to edge spacing of adjacent nanowells may be based on the optical resolution of the detection system in order to prevent cross-talk between adjacent nanowells 200. In embodiments, the nanowells may have a depth below the top surface 201 between 50 nm and 300 nm, for example 100 nm.

Each nanowell 200 may include a derivitized area 102 for binding to sample nucleic acid molecules, as discussed above in relation to FIGS. 1A-1C. As shown in FIG. 2B, the derivitized area 102 may be defined on the bottom surface of the nanowell 200. In embodiments, the derivitized area 102 may encompass the entire bottom surface of the nanowell 200, or only a portion thereof. In embodiments, the derivitized area 102 may include one or more portions on sidewalls of the nanowell 200.

The derivitized area 102 in addition to the physical structure of the nanowell 200 may both prevent the DNBs from spreading out beyond the nanowell. For example, as discussed above, DNBs may flatten once bound to a derivitized area. As shown in FIG. 2C, while flattening out the DNB 103 may contact the sidewalls 204 nanowell 200, and instead of continuing to spread radially beyond the derivitized area, as shown on the planar substrate in FIGS. 1C and 1D, the DNB is contained within the nanowell, and thus the nanowells increase the surface distance between adjacent derivitized area compared to a planar substrate. This containment prevents adjacent DNBs from contacting, even if the DNB is grown after being bound to the derivitized area. Accordingly, nanowells prevent contamination and cross-talk, as discussed above relating to FIGS. 1A-1D. In embodiments, the volume of each nanowell 200 is selected to be greater than a predetermined maximum volume of a growing DNBs.

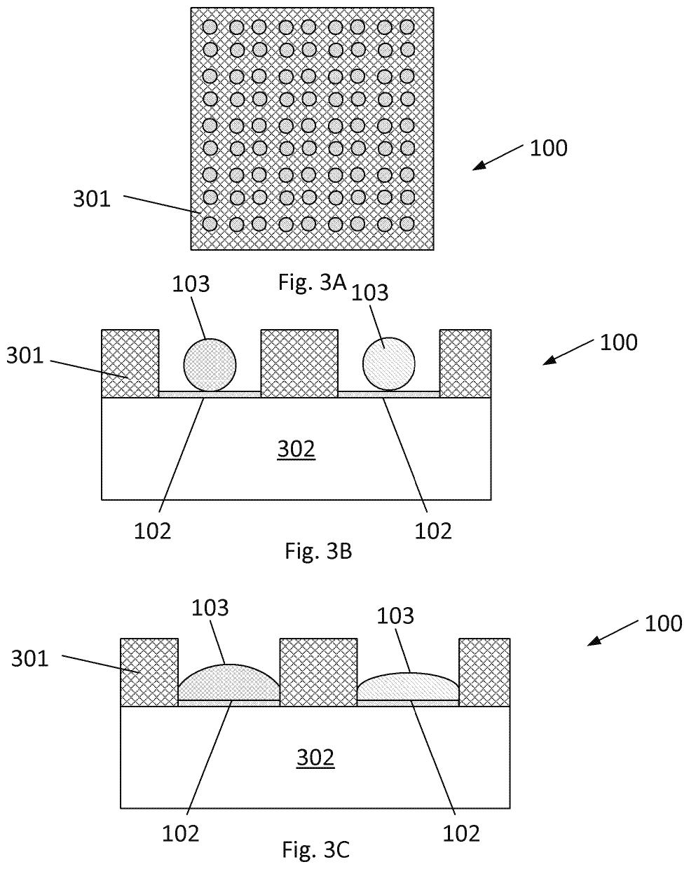

In embodiments, the nanowells 200 may be formed as part of a substrate 100 with one or more of a plurality of manufacturing methods, including, but not limited to: microlithography, photolithography, soft lithography, and nanoimprint lithography. In embodiments, as shown in FIGS. 3A-3C, the nanowells 200 may be formed with a lattice of one or more layer of material on top of a substrate base. In embodiments, the lattice may be partially or fully formed of one or more layers, for example metal oxide or nitride layers. In embodiments, the lattice is formed of one or more layers including layers of organic material, for example photoresist 301, on top of a layer for binding to photoresist on a substrate base 302. The substrate base may be formed of a single material, or a plurality of layers of different material. The lattice may be formed by applying a layer of organic material, e.g. photoresist, on top of the substrate base 302, followed by exposing the organic material to a patterned light, and developing the exposed organic material to remove portions of photoresist to define the nanowells. As shown in FIGS. 3B and 3C, the bottom of the nanowells may be defined by the base substrate 302, and the sidewalls may be defined by the organic material. In embodiments, the derivitized areas of each nanowell may be defined, e.g. with surface chemistry, before or after the patterning of the nanowells.

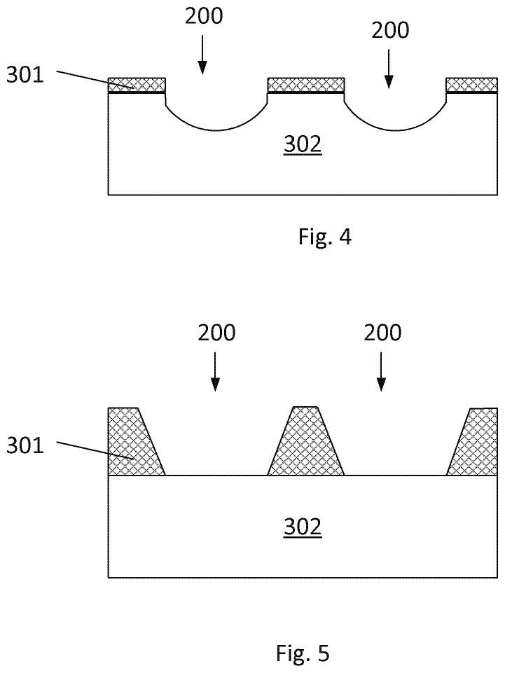

In embodiments, the nanowells 200 may be formed, at least partially into the base substrate. For example as shown in FIG. 4, nanowells may be formed by exposing the organic material on the top surface of the base substrate to a patterned light, the organic material is then developed to remove portions of organic material, e.g. photoresist, over the locations of the nanowells, and then the uncovered base substrate previously under the removed organic material may be etched to remove portions of the base substrate to define at least a portion of the nanowells.

In embodiments, as shown for example in FIGS. 3C, the bottom surface of the nanowells may be substantially flat, and in embodiments, the bottom surface of the nanowells may be curved, for example as shown in FIG. 4. In embodiments, as shown for example in FIG. 3C, the sidewalls of the nanowells may be substantially perpendicular relative to the top surface, and in embodiments, the sidewalls of the nanowells may be curved or angled relative to the top surface, for example as shown in FIG. 5. The shape of the bottom surface and sidewalls may be selected to maximize the reflecting of optical signals toward a detection system to detect sequencing events in the nanowells. In embodiments, the bottom surface and/or sidewalls may be parabolic in shape to reflect and focus optical signals from the DNBs to the detection system.

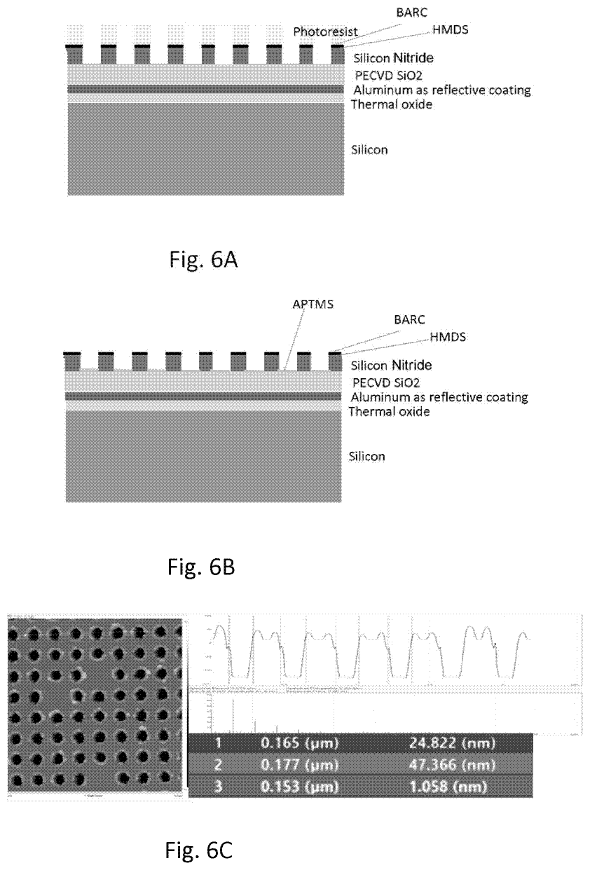

In some embodiments, for example as shown in FIGS. 6A and 6B the nanowells may be formed in a processed referred to as Non-binding BARC-Binding Silicon Nitride/APTMS using Photolithography. In this process, the nanowell structure may be may fabricated with photoresist (organic material) over an oxide/Silicon Nitride binding layer over a SiO2/aluminum reflective layer over a silicon base layer, as shown in FIG. 6A. The top surface structure of FIG. 6A may be aminated by APTMS followed by a stripping of the photoresist to remove the top photoresist resulting in the structure shown in FIG. 6B. This process results in the bottom surface of each nanowell being coated with APTMS as binding material. As shown in FIG. 6A, a BARC layer (organic material) may be present between the photoresist and the HMDS layer on top of the Silicon Nitride. After the striping of the photoresist, the BARC layer (organic material) will remain as the top exposed around each of the nanowells. The BARC layer is a “non-binding” material. The BARC layer stands for bottom anti-reflective coating, and is beneficial in that it may be used for photolithography process for high density features to reduce the reflectivity during the exposure as well as being a non-binding material for DNBs. FIG. 6C shows an AFM image of nanonwells with a 360 nm pitch corresponding to FIG. 6B using Silicon Nitride/APTMS as binding surface and BARC layer as non-binding surface.

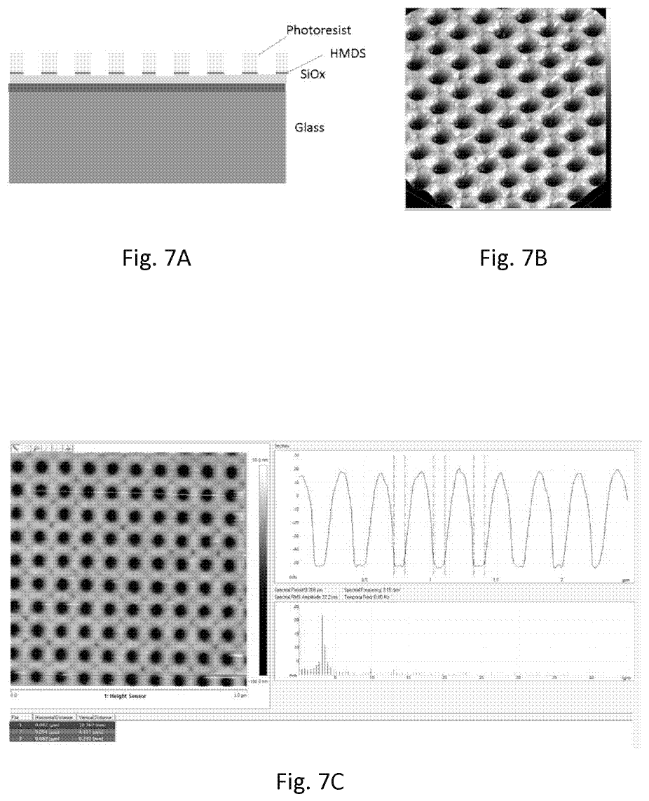

In some embodiments, for example as shown in FIG. 7A nanowells may be formed in a processed referred to as Non-binding Resist-Binding Silicon Oxide using Photolithography. In this process, nanowells are fabricated with non-binding resist (organic material) over a binding SiOx layer over a reflective layer over a base, in the example shown glass. In this approach, there is no stripping of the photoresist as the binding layer of the nanowells is formed prior to adding the photoresist, and the photoresist itself forms sidewall of the nanowells. This process is different than the process of in FIGS. 6A and 6B wherein the photoresist is used in the patterning of the Silicon Nitride forming the sidewalls of the nanowells as well as part of the process of forming binding surfaces at the bottoms of the nanowells but not on the top surfaces around the nanowells.

FIGS. 7B and 7C show atomic force microscopy (AFM) images of substrates including nanowells formed of lattices of material with a 360 nm pitch, e.g. photoresist, on top of a base substrate corresponding to FIG. 7A.

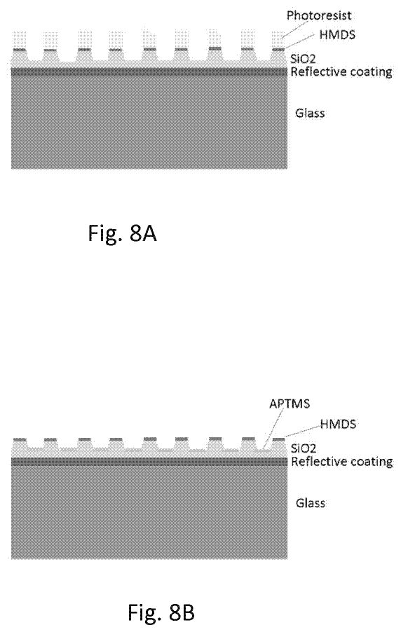

In some embodiments, for example as shown in FIGS. 8A and 8B nanowells may be formed in a processed referred to an Non-binding HMDS/Binding APTMS using Nanoimprint Lithography. Nanoimprint lithography may include two separated two steps: the fabrication of a patterned hard mold and the use of this mold to transfer the pattern into a polymer film for nanoscale patterning. The nanowell structures may be imprinted in a UV-curable resist (organic material) over SiO2 by applying a broadband UV light radiation directly through the backside of the transparent mold causing the resist monomer crosslink to form a rigid polymer. After releasing the mold, a SiO2 may be dry etched resulting in nanowells, as shown in FIG. 8A. After this formation of the nanowells, the top surface of the structure of FIG. 8A, including the photoresist may be aminated by APTMS. Following the aminating, the photoresist may be strips resulting in the structure shown in FIG. 8B. The bottom surface of the nanowells remains coated with APTMS as binding material after the photoresist is stripped. An HMDS layer (organic material) may be present on top of the SiO2 and will be exposed after the photoresist stripping around each nanowell structure as a “non-binding” material. In this approach, the pitches being nanowells may be in a high density range of 250 nm to 300 nm.

The substrate and/or base substrate, for example as shown in FIGS. 1A-6B, may be formed of one or more layers. In embodiments, the base substrate may include a combination of one or more layers of: silicon, hexamethyldisilazane (HMDS), aluminum, oxides, organic material (e.g. photoresist). Oxides, such as Titanium oxide (TiOx), Silicon oxide (SiO2), Tetraethyl othosilicate (TEOS), may form binding surfaces at the bottoms of nanowells. Aluminum, Titanium, or Chromium layers may act as reflective coating for reflecting the DNB fluorescence signal from the surface as signal enhancement layer. As noted above, photoresist may be included in the final nanowell structure and be used as non-binding surface outside the nanowells.

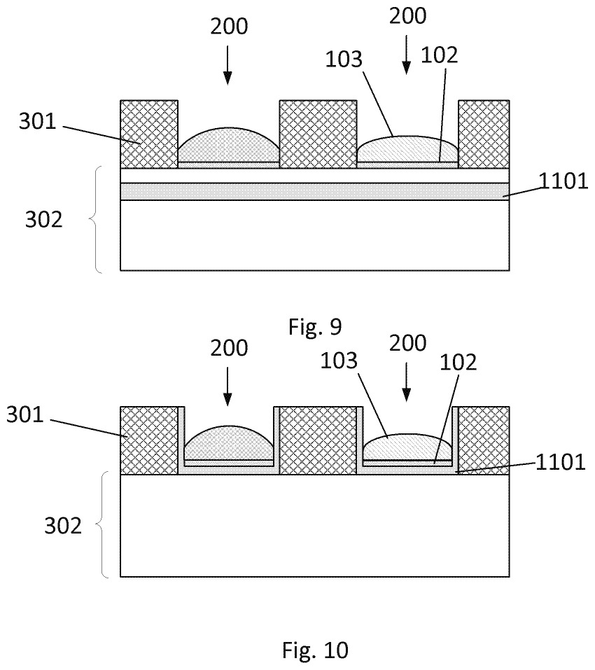

In embodiments, the detected brightness of a DNB corresponds to the number of copies of the nucleic acid molecules comprising the DNB, which corresponds to the volume of the DNB. The present nanowell technology in addition to increasing the number of derivitized areas per substrate area by allowing closer spacing of adjacent derivitized areas relative to planar substrates, may also have increased number of derivitized areas per substrate area by having smaller derivitized areas relative to planar substrates. Since the brightness of a DNBs at a derivitized area corresponds to the number of copies of the nucleic acid molecules, which corresponds to the volume of the DNB, in embodiments it is beneficial to increase the detected brightness of DNBs on a substrate by including one or more reflective portions on the substrate, for example a reflective layer in the base substrate, or reflective walls (e.g. metalized walls) of each nanowell. The reflective portions may be composed of metal or a metal oxide, for example, Aluminum, Chromium, and Titanium. In some embodiments, the reflective portion may a dielectric stack of materials (at least 2 or 4) with alternating refractive indices such that the stack forms a dielectric mirror. This may be similar to an anti-reflective coating applied to some substrates or lenses, except for replacing destructive interference with constructive interference. In embodiments, the substrate 201 or base substrate 302, for example as shown in FIGS. 1A-6B, may include a reflective layer 1001 under derivitized areas or under the nanowells, for example as shown in FIG. 9. In embodiments, one or more of the sidewalls and/or bottom surface of each nanowell may include a reflective surface 1101, for example as shown in FIG. 10. Reflective portions may increase the detected brightness of DNBs due to increased input excitation laser light reaching the DNBs. The increased input excitation laser light is due to portions of the input excitation laser light which would otherwise pass the DNB and not cause emissions being reflected back toward the DNB to cause increased emissions. Reflective portions may further increase the detected brightness of DNBs due to the reflective portions focusing more of the emission from the DNBs toward the detection system, compared to substrates without reflective portions. In embodiments, substrates including reflective layers may result in the detected brightness of DNBs increasing 2-3 fold relative to substrates without reflective surfaces.

In embodiments, one or more of the layers of the substrate may include plasmonic enhancement structures. For example a layer of SiO2 under the nanowells may include metal grains tuned to couple photos into surface plasmons, which results in strong optical signals. In some embodiments, plasmonic enhancement structures results in 4× brightness increase in the green channels and 14× increase in the red channels during imaging of sequencing. In some embodiments, plasmonic coatings for signal enhancement material may be added to a nanowell substrate structure. In general, plasmons are collective excitation of free electrons in metal nano particle (e.g. Silver and gold). When the free electrons are stimulated by an energy source like a laser, the nanoparticles set up harmonic oscillations of the surface charges in the metal atom.

The three-dimensionally patterned substrates, including nanowells, as disclosed herein may be part of a flowcell of a sequencing system, wherein the nucleic acid template molecules (e.g., DNBs) may be immobilized in the nanowells prior to or after incorporating the substrate into the flow cell. During a sequencing procedure, wash buffers may be separately flowed through the flowcell and over the substrate. Due to the closer spacing of the derivitized areas in substrates including nanowells, reagents flowed into the flow cell will react with nucleic acid template molecules at more derivitized areas than a flowcell without nanowells, and therefore less reagent may be used per derivitized area.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

It is to be understood that the above description is intended to be illustrative and not restrictive. Many embodiments will be apparent to those of skill in the art upon reviewing the above description. The scope of the invention should, therefore, be determined not with reference to the above description, but instead should be determined with reference to the appended claims along with their full scope of equivalents.

While the foregoing disclosure shows illustrative aspects of the disclosure, it should be noted that various changes and modifications could be made herein without departing from the scope of the disclosure as defined by the appended claims. The functions, steps and/or actions of the method claims in accordance with the aspects of the disclosure described herein need not be performed in any particular order. Furthermore, although elements of the disclosure may be described or claimed in the singular, the plural is contemplated unless limitation to the singular is explicitly stated.

1. A method of preparing a three-dimensionally patterned substrate for nucleic acid sequencing, the method comprising:
providing a planar substrate;
defining a plurality of nanowells recessed below a top surface of the planar substrate, wherein the nanowells are arranged in an array and wherein the planar substrate contains plasmonic enhancement structures positioned at least in a portion of the substrate below the nanowells; and
defining a surface chemistry so that each nanowell comprises a binding surface comprising surface chemistry configured to bind to template nucleic acid molecules.

2. The method of claim 1, wherein the planar substrate comprises a reflective portion.

3. The method of claim 2, wherein the reflective portion comprises an aluminum, chromium, or titanium portion.

4. The method of claim 2, wherein the reflective portion comprises a reflective layer positioned between bottom surfaces of the plurality of nanowells and a layer of the planar substrate comprising silicon; and wherein the plasmonic enhancement structures are located in the layer of the planar substrate comprising silicon.

5. The method of claim 2, wherein the reflective portion comprises reflective walls of each of the nanowells.

6. The method of claim 5, wherein the reflective walls comprise metalized walls.

7. The method of claim 2, wherein the reflective portion is configured to reflect excitation light into the nanowells, and reflect emission light from the nanowells upwardly.

8. (canceled)

9. The method of claim 1, wherein the plasmonic enhancement structures comprise metal grains in an oxide layer of the planar substrate below the nanowells.

10. The method of claim 9, wherein the plasmonic enhancement structures are configured to be tuned to couple photons into surface plasmons.

11. The method of claim 1, wherein the substrate comprises a base substrate and a layer of organic material on a top surface of the base substrate, and
wherein defining the plurality of nanowells comprising removing portions of the organic material.

12. The method of claim 11, wherein the organic material comprises photoresist, and removing portions of the organic material comprising performing a photolithography process to the photoresist.

13. The method of claim 12, wherein removing the portions of the organic material comprises forming a lattice of the organic material, and wherein voids in the lattice define the nanowells.

14. The method of claim 1, wherein the substrate comprises a base substrate and a layer of organic material on a top surface of the base substrate, and
wherein defining the plurality of nanowells comprises removing portions of the organic material and the base substrate.

15. The method of claim 14, wherein the base substrate comprises silicon or glass and the organic material comprises photoresist.

16. The method of claim 1, wherein each of the plurality of nanowells are defined to comprise a flat bottom surface.

17. The method of claim 1, wherein each of the plurality of nanowells are defined to comprise a curved bottom surface, and wherein each of the curved bottom surfaces are configured to focus emissions upwardly out of the nanowells.

18. The method of claim 17, wherein the curved bottom surface is parabolic in shape.

19. The method of claim 1, wherein the nanowells satisfy at least one condition of:
each nanowell defining a diameter of less than 300 nm;
a center-to-center spacing of the plurality of nanowells in the array being less than 500 nm; and
plurality of nanowells each defining a depth of less than 200 nm.

20. The method of claim 19, wherein each nanowell defines a diameter of less than 150 nm.

21. (canceled)

22. The method of claim 19, wherein a center-to-center spacing of the plurality of nanowells in the array is less than 350 nm.

23. (canceled)

24. The method of claim 19, wherein the plurality of nanowells each define a depth of less than 100 nm.

25. The method of claim 24, wherein the plurality of nanowells each define a depth between 60 nm and 100 nm.

26-48. (canceled)
